(19) United States
(12) Patent Application Publication
(10) Pub. No.: US 2002/0104126 A1
(43) Pub. Date: Aug. 1, 2002

(54) Production of bovine lysozyme by plant viral vectors

(76) Inventors: Gregory P. Pogue, Vacaville, CA (US); N. Sharlene Velichko, Vacaville, CA (US)

Correspondence Address: Dean H. Nakamura, Roylance Abrams Berdo & Goodman, 1300 19th Street, NW, Washington, DC, 20036, US

(21) Appl. No.: 09/978,199

(22) Filed: Oct. 17, 2001

Related U.S. Application Data
(60) Provisional application No. 60/240,967, filed on Oct. 18, 2000.

(51) Int. Cl. A01H 5/00; C12N 7/00; C07H 21/04; C12N 15/86

(52) U.S. Cl. 800/288; 435/235.1; 435/320.1; 536/23.2

(57) ABSTRACT

Bovine lysozyme is produced in plants.

### BACKGROUND OF THE INVENTION

1. Field of the Invention

The present invention is directed to viral vectors and methods for producing transgenic plants that express heterologous DNA that encode a ruminant lysozyme, preferably bovine. This lysozyme protects against diseases caused by plant pathogens, particularly bacterial pathogens. This invention is also directed to methods for producing a ruminant lysozyme from transgenic plants that express the lysozyme.

2. Description of the Background Art

Varieties of the gram-negative plant pathogenic bacterium Xylella fastidiosa cause a range of diseases in economically important crops. These diseases include Pierce's disease (“PD”) in grapevines, alfalfa dwarf, phony peach disease, periwinkle wilt and leaf scorch of plum (and oak). A cloned strain of Xylella fastidiosa induced symptoms of citrus variegated chlorosis in sweet orange (Li WB et al, Curr Microbiol. 39:106-108 (1999 August).

PD, spread by sharpshooter leafhoppers, threatens the vine industry of California. PD is only known from North America through Central America and has been reported in parts of northwestern South America. Xylella fastidiosa is a highly specialized pathogen responsible for this disease. It multiplies in the foregut of sharpshooter leafhoppers, which feed on sap in a plant's xylem, the main water-conducting tissue. The insect carrier delivers bacteria directly into the xylem system of host plants. There they multiply, forming a polysaccharide matrix within the xylem, which eventually blocks the flow of nutrients and results in tissue death. Symptoms include chlorosis and premature production of fruits which are small, tough and, therefore, worthless. The disease is potentially devastating; the most effective control is to produce healthy bacteria-free material for plant propagation. As noted, other strains of the pathogen cause far ranging disease in citrus, nut, fruit and other ornamental plants cultivated in the Western Hemisphere.

The bacterium that causes PD is limited to the xylem. Insects with piercing/sucking mouthparts that feed on xylem sap transmit the bacteria from diseased to healthy plants. Vines develop symptoms when the bacteria block the water conducting system and reduce the flow of water to affected leaves. Water stress begins in mid-summer and increases through fall. The first evidence of PD infection usually is a drying or “scorching” of leaves. The leaves become slightly yellowed (chlorotic) along the margins before drying, or the outer leaf may dry suddenly while still green. Typically, the leaf dries progressively over a period of days to weeks, leaving a series of concentric zones of discolored and dead tissue. About mid-growing season, when foliar scorching begins, some or all of the fruit clusters may wilt and dry up. “Scorched” leaves detach from the distal end of the petiole (leaf stem) rather than from the base of the petiole, leaving the bare petioles attached to canes, often well after normal leaf fall. The bark on affected canes often matures unevenly, leaving islands of mature (brown) bark surrounded by immature (green) bark or the reverse.

A major effort by a Brazilian consortium resulted in the sequencing of the entire Xylella genome (citrus strain), representing the first plant bacterial pathogen to be characterized to such detail (Silvestri, M. L et al. Nature 406:151-157 (13 July 2000); See, also, Frohme M et al., Nucleic Acids Res. 28:3100-3104 (15 Aug 2000). The gene complement of X. fastidiosa reflects its life in plant xylem in three main ways. The bacterium has become adapted to use a variety of free sugars found in xylem sap, and to supplement these sugars with glucose derived from the breakdown of cellulose, the main component of plant cell walls. Adding to the picture of a honed-down metabolism, genes encoding the enzymes needed to make sugars from amino acids and other metabolites are missing; this shows that the organism has a strict requirement for carbohydrates as the sole energy source and source of building blocks for all biosynthetic reactions. No fewer than 67 genes are devoted to the uptake of iron and other transition metals from xylem sap, and it has been suggested that depletion of these micronutrients contributes to symptoms of infection. Third, X. fastidiosa produces two distinct cell-adhesion systems. One comprises a matrix of extracellular polysaccharides, synthesized by the appropriately named gum genes that embed the bacteria in a matrix in the xylem, eventually leading to blockage of xylem flow and host water-stress. The other system is for bacterial adhesion to the gut and mouthparts of the insect vector, and is specified by 26 genes encoding the so-called fimbral proteins that are needed for bacterial adhesion and translocation across cell surfaces.

In the past, modified agronomic practices controlled the distribution of the common, vector, the blue-green sharpshooter, and were sufficient to prevent chances of large-scale loss of vines to PD. This was possible due to the limited feeding and migration habits of the indigenous sharpshooters and their habitat preferences. However the migration of a new, more challenging, vector, the glassy-winged sharpshooter, from Ventura and across the Mexican border into California has changed the entire equation. This vector is physically larger and exhibits different feeding habits than its more diminutive blue-green counterpart. The glassy-winged sharpshooter feeds much lower on the can than most indigenous California sharpshooters. It feeds later in the season resulting in infections to grape that can survive winter and continue the disease through the next season. The sharpshooter also exhibits winter-feeding that will encourage vine-to-vine transmission. One additional trait of this sharpshooter that makes it problematic is its promiscuous feeding habits. It feeds on many herbaceous and tree species propagated for ornamental and orchard purposes. This leads to an extensive set of hosts that can be carriers for the Xylella pathogen. Feeding of populations of sharpshooters results in transmission and infection between many species that are growing in the vicinity of an infected plant. Indeed, many worst-case predictions have proved true. The glassy winged sharpshooter has rapidly migrated from the Temecula Valley to virtually every county in California that propagates vines. The presence of the vector and the ubiquitous presence of potential carrier hosts for the bacterium present a large challenge to the entire California wine industry as well as growers of citrus and other valuable host plants.

Lysozymes (β1-4-N-acetylmuramidase enzymes

Lysozymes are a class of antimicrobial proteins encoded by diverse organisms ranging from bacterial T-even phages to higher vertebrates. Lysozymes vary in molecular masses from 14 kDa (egg white) to 18 kDa (bovine milk). The peptidoglycan backbone of the bacterial cell wall can be cleaved by lysozymes, a ubiquitous family of enzymes in many tissues and secretions of humans, other vertebrates and invertebrates, as well as in plants, yeast (e.g., Pichia pastoris; Digan et al., Bio/Technology 7:160-164 (1989)) bacteria and phage. Lysozymes are basic enzymes that catalyze the hydrolysis of the β(1-4)glycosidic bond between the C-I of N-acetylmuramic acid and the C-4 of N-acetylglucosamine (“GlcNAc”), which occurs in the component of bacterial cell walls, bacterial peptidoglycan or murein.

Types of Lysozymes

Several types of lysozymes that differ based upon their amino acid sequence and structure, have been identified. Lysozymes of the c, or “chicken”, type contain 129-130 amino acids in their mature, secreted forms. About 40 of the 129-130 amino acids appear to be invariant among different species. Eight Cys residues of all of the c-type lysozymes are invariant. The disulfide bonds formed by these residues are important in the formation and maintenance of the secondary and tertiary structures of the lysozymes, which appear to be similar for all type c lysozymes. The complete primary structures are known for the mature lysozyme c from numerous sources, including (1) hen egg white, quail, turkey, guinea fowl, duck and pheasant; (2) human milk and urine; (3) moth; (4) baboon; (5) rat; and (6) bovine stomach. The sequence of DNA that encodes mature human milk lysozyme c is also known. See European Patent Application Publication Nos. 0 181 634, 0 208 472, and 0 222 366.

The g-type lysozymes contain about 185 amino acids in their mature forms, exhibit low activity on GlcNAc polymers, and do not cross-react immunologically with lysozymes of the c-type. G-type lysozymes have an unusually high occurrence of paired amino acids, in which the same amino acid occurs at neighboring positions in the molecules, and all of the four Cys residues are situated in the N-terminal half. G-type enzymes only hydrolyze, whereas, c-type lysozymes can hydrolyze and transglycosylate. The existence of other distinct types of lysozymes, which differ from the c and g types on the basis of structural, catalytic and immunological criteria, has also been reported. Bacteriophage lysozymes, such as T2 and T4 phage lysozymes, include 164 amino acids and have a molecular weight of 18,700. A lysozyme activity has been detected in several plant tissues, but the plant lysozyme appears to act as a chitinase rather than as a 1,4-β-N-acetylmuramidase.

In addition, lysozymes may also be characterized on the basis of their in vivo biological activity. Because of their ability to cleave the peptidoglycan bacterial cell wall, some lysozymes are involved in mammalian defense systems. A number of mammalian species, which have a foregut or rumen, have an unusually high level of lysozyme in the fundus region of the stomach. This lysozyme, which has a unique activity profile, appears to have evolved to perform functions distinct from other lysozymes. Most lysozymes appear to function to protect against infection and in other defense systems. Ruminant stomach lysozymes, however, have evolved as digestive enzymes to digest the microbes that grow in the foregut and thereby scavenge the nutrients used by these microbes that digest cellulose.

Ruminant Gut Lysozymes

A ruminant is a cud-chewing mammal with two stomachs: a foregut in which anaerobic gram-positive bacteria digest cellulose, thereby permitting the ruminant to use cellulose as a source of energy and nutrients; and a true stomach. Ruminants, such as domestic cattle and other cud-chewing mammals in the order Artiodactyla have developed a symbiotic relationship with bacteria that live in the rumen thereby permitting ruminants to use cellulose as a major nutrient. The bacteria digest cellulose and other dietary components and rapidly grow and divide to large numbers. They convert a significant percentage of the nutrients that are ingested by the ruminant.

Lysozymes digest the cell walls of the microbes under the acidic conditions in the true stomach of ruminants and ruminant-like species, including cows, sheep and deer, have evolved (Dobson et al. (1984) J. Biol. Chem. 259:11607-11616). The lysozymes digest the bacteria, enabling the ruminant to utilize the lysed bacteria as a source of carbon, nitrogen, and phosphorus for energy and growth. Bovine stomach lysozyme was first purified from mucosa by Dobson et al. ((1984) J. Biol. Chem. 259:11607-11616). Three distinct, related, non-allelic forms of lysozyme c were isolated and constitute approximately 10% of the total protein that can be extracted from the ruminant stomach mucosa. These three nonallelic lysozymes, designated c1, c2 and c3, are closely related to one another antigenically and in amino acid composition. These type c lysozymes have functionally diverged from other mammalian lysozymes: their pH optimum (for enzymatic activity) is approximately 5, rather than 7 as exhibited by other type c lysozymes, rendering them more stable in acidic environments and more resistant to proteolytic enzymes, such as pepsin (see Jolles et al. (1984) J. Biol. Chem. 259:11617-11625). Several poperties, including the complete 129 amino acid sequence of a mature bovine lysozyme c2 indicate that this enzyme diverged from other lysozymes (Jolles et al., supra) Ruminant lysozyme c, a digestive enzyme, permits ruminants to use the lysed bacteria as sources of carbon, nitrogen and phosphorous. This lysozyme is confined to the stomach; it has not been found in other tissues or secretions. In non-ruminant species, stomach lysozymes appear to be identical with the lysozymes in other tissues and secretions.

Lysozymes as Anti-Bacterial Agents

Lysozymes exhibit anti-bacterial activity. When used as antimicrobial agents, lysozymes are generally employed in combination with other agents, such as lacto-transferrin, complement, antibodies, vitamins, other enzymes and various antibiotics, including tetracycline and bacitracin. Such antimicrobial compositions are used as preservatives for dairy and meat products and marine products.

Antimicrobial compositions that contain ruminant lysozyme c and endo-β-N-acetylglucosaminidase or endoglycopeptidase that are formulated as mouthwashes, soaps, contact lens cleaners and other similar products are described in Europe Patent Application 042019A1 (Feb. 5, 1991) to The Proctor & Gamble Company) discloses the lysozyme c is not sufficiently effective against certain bacteria in certain sites in mammals to be used alone. Thus, disclosed antibacterial compositions include the endo-β-N-acetylglucosaminidase or endoglycopeptidase in addition to the lysozyme. Lysozymes are purportedly not effective against gram-negative bacteria.

Thus, lysozyme appears to be most useful, when used as an antimicrobial agent, for lysing gram-positive bacteria. It has been suggested (U.S. Pat. No. 5,850,025) that lysozyme was not an agent of plants that are plagued by diseases caused by gram-negative organisms. The majority of bacterial plant pathogens are gram-negative. In addition, c-lysozymes are unstable, particularly under conditions in which plants are grown or under which seeds are stored. Therefore, it was stated (ibid) that lysozymes are unsuitable for treating plants due to the nature of most bacterial pathogens and the instability of lysozymes.

According to U.S. Pat. No. 5,850,025), (1) there were few means for controlling plant bacterial pathogens, (2) those that were available (e.g., heavy metal-containing sprays and antibiotics), were not highly effective and were environmentally unacceptable, and (3) since there were relatively few bacteria-resistant vegetable or fruit plants available, there was a need for the development of effective, non-toxic, biodegradable and environmentally acceptable means for the control of plant pathogens. The foregoing patent and its parent (U.S. Pat. No. 5,422,108) disclosed methods for treating plants, plant tissues, and seeds infected with plant pathogens and for treating non-infected plants to render them resistant to or less susceptible to infection by plant pathogens. These patents disclosed methods for protecting plants against bacterial infection by contacting the plants with a composition that contains an effective concentration of a lysozyme that is sufficiently stable to protect the plant against this infection. The ruminant lysozyme, ruminant-like lysozyme or mixture was sprayed on crop plants in the field or in a greenhouse or the seeds coated with the lysozyme.

DNA encoding a lysozyme is used for constructing transgenic plants that are of reduced susceptibility or are resistant to particular plant pathogens or for preparing host cells that are cultured in vitro in order to produce the lysozyme for use in treating plants, plant tissues and seeds or to replicate DNA for use in preparing transgenic plants.

U.S. Pat. Nos. 5,422,108 and 5,850,025 disclosed use of DNA encoding a lysozyme for (1) constructing transgenic plants that have reduced susceptibility or are resistant to particular plant pathogens or (2) for preparing host cells that are cultured in vitro in order to produce the lysozyme for use in treating plants, plant tissues and seeds or to replicate DNA for use in preparing transgenic plants. The documents stated that any lysozyme and DNA encoding it such that possesses the requisite stability and activity against gram-negative bacteria were suitable for use. The lysozyme may be purified tissue or prepared recombinantly. The protein was said to be a naturally occurring ruminant or ruminant-like lysozyme, such as bovine lysozyme c2, or a modified form of a ruminant or ruminant-like lysozyme, such as lysozyme encoded by DNA that was prepared synthetically or produced by point mutation, insertion or deletion of naturally occurring, cloned or synthetic DNA (citing Dobson et al., supra).

Killing of Xylella

Unlike other disclosed plant pathogenic bacteria, Xylella fastidiosa are xylem-limited pathogens. There was therefore a need in the art to make lysozyme and target it to affected plants so that it can kill Xylella fastidiosa in valuable crop plants.

### SUMMARY OF THE INVENTION

This invention is based in part on the observation that lysozyme from bovine stomach mucosa shows broad activity on Gram-positive and Gram-negative bacteria and significant chitinase activity (US Pat. No. 5,422,108; Pahud and Widmer, 1982, Biochem. J. 201:661). Activity was observed against Gram-negative bacteria was observed at concentrations as low as 25 ppm. The pH optimum for bovine stomach lysozyme (“bolys”) ranges between 4 and 6 and the enzyme shows remarkable pH and heat stability. Due to the broad anti-bacterial actions of these enzymes, their ability to kill Gram-negative bacteria, their stability and their activity optima, this enzyme was selected by the present inventors as an anti-Xylella reagent for development of a recombinant source of the bolys protein for treating vines to prevent or treat PD.

Bovine lysozyme is useful in protecting grapevines from destruction by Xylella fastidiosa. A recombinant virus containing a nucleic acid encoding bovine lysozyme is shown to be useful in causing a host plant to produce bovine lysozyme.

The present invention provides a method of making bovine lysozyme in a plant by infecting a host plant with such a recombinant virus. This method is used, for example, to protect grapevines.

This method has the advantage that it results in rapid and economic production of bovine lysozyme. A method of isolating such virally encoded bovine lysozyme from a host plant makes this enzyme readily available for protecting grapevines from Xylella infection.

This invention provides a plasmid encoding a recombinant RNA plant virus, which comprises a bovine lysozyme encoding nucleic acid (preferably SEQ ID NO:1) or a biologically active variant thereof. A preferred plasmid has the sequence SEQ ID NO:3 (see FIG. 2).

This invention also provides a recombinant RNA plant virus comprising a bovine lysozyme encoding nucleotide sequence (preferably SEQ ID NO:1).

This invention provides an RNA sequence, comprising a first and a second viral subgenomic promoter and a bovine lysozyme coding sequence under control of either the first or the second subgenomic promoter.

This invention provides a recombinant Tobamovirus comprising a bovine lysozyme encoding nucleic acid sequence.

This invention provides a method of making bovine lysozyme and of isolating bovine lysozyme from a plant.

### BRIEF DESCRIPTION OF THE DRAWINGS

FIG. 1 shows the nucleotide sequence encoding bovine lysozyme (SEQ ID NO: 1) and the encoded bovine lysozyme protein (SEQ ID NO:2). An 18 amino acid signal sequence is cleaved between G and K residues.

FIG. 2 shows the nucleic acid sequence of the vector p1044 BoLys (SEQ ID NO:3) which includes SEQ ID NO: 1 encoding bovine lysozyme inserted as a PacI-XhoI fragment.

FIG. 3/2 is a schematic illustration of the p0144-BoLys vector. The genome of the tobacco mosaic virus is diagrammatically shown to reveal replicase coding regions (126 and 183 kDa proteins), movement protein (30 kDa), bovine lysozyme (bolys) and non-native coat protein (hCP). Regulatory elements known as subgenomic promoters are shown: SP-E transcribes the mRNA for the 30 kDa gene early in infection. SP-1 transcribes mRNA encoding Bolys protein. SP-2 transcribes mRNA encoding coat protein.

FIG. 4 is an SDS-PAGE gel showing total plant extracts from 3 independent Nicotiana benthamiana plants inoculated with p1044 BoLys. Molecular weight markers are shown in lane 1 followed by increasing amounts of purified bolys. In each of lanes 5-7, 2 μl of total soluble proteins from individual plants were loaded. The location of TMV coat protein and bolys protein are indicated by arrows on the right. Higher molecular weight proteins and protein band slightly smaller than bolys represent major plant proteins involved in light harvesting or energy transfer.

FIG. 5 is an SDS-PAGE gel showing IF plant extracts from 2 independent batches of Nicotiana benthamiana plants inoculated with p¹⁰⁴⁴ BoLys. Molecular weight markers are shown in lane 1 and 12. Increasing amounts of purified bolys (provided by NewGene) are shown in lanes 3-7. Hen egg lysozyme is shown in lane 2 for comparison purposes. Different volumes of IF extract (apoplastic protein fraction) from two independent batches of plants (>300 g each) are shown in lanes 8-11. Migration positions of TMV coat protein and bolys protein are indicated with arrows on the right side.

FIG. 6 shows mass spectrographic (MALDI-TOF) analysis of crude IF from p1044 BoLys infected Nicotiana benthamiana plants.

FIG. 7 shows results of turbidimetric assay of bovine lysozyme activity comparing a standard with a partially purified preparation derived from plants infected with p1044BoLys which were extracted and the extract subjected to ultrafiltration to obtain material having a molecular mass over 3 kDa.

FIG. 8 shows results of turbidimetric assay of bovine lysozyme activity. Assays were carried out using purified bovine lysozyme provided by NewGene spiked into IF plant fluid from healthy plants and crude IF fluid isolated from plants infected with p1044BoLys. Assay conditions are described elsewhere. The general results over several experiments are summarized as follows:

| | |
|---|---|
| Positive Control BoLys: | ˜7000 units/mg |
| Crude IF BoLys: | ˜8000 units/mg |

FIG. 9 shows results of a fluorometric assay of bovine lysozyme activity. The assay was carried out using purified bovine lysozyme provided by NewGene spiked into plant extracts from healthy plants and extracts isolated from plants infected with p1044BoLys. Assay conditions are described elsewhere.

FIG. 10 is a graph showing turbidity of various samples (different concentrations of enzyme preparation) over time.

### DESCRIPTION OF THE PREFERRED EMBODIMENTS

The bovine lysozyme (bolys) gene was obtained from Erik Mirkov (Texas A&M University) and oligonucleotides were designed that allowed amplification (by polymerase chain reaction—PCR) of the gene from the donor plasmid with restriction sites at either end of the PCR product. The gene fragment was digested with appropriate enzymes (Pac I and Xho I) and ligated into the LSBC GENEWARE® vector pBTI 735 that was similarly digested to yield p1044 BoLys. GENEWARE refers to a family of expression vectors based on virus genome components and that are capable of autonomous amplification. The resulting ligation was transformed into DH5 alpha E. coli cells and resulting colonies were picked, grown and the DNA purified. The purified DNA was then subjected to digestion to map successful ligation of the gene product and clones deemed positive by this screen were confirmed by DNA sequencing of the entire bolys gene. All clones that contained the bolys gene included the bovine lysozyme DNA sequences present in GenBank.

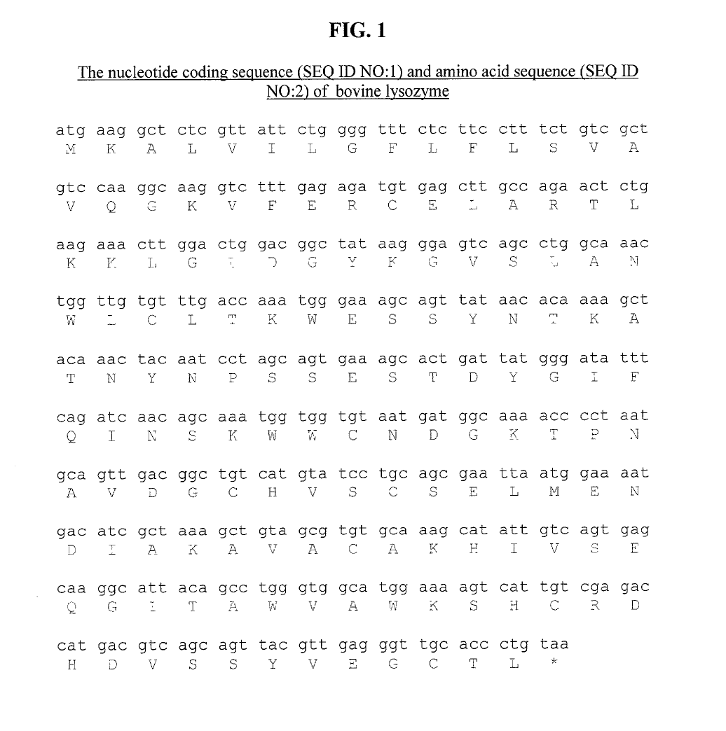

The bolys gene encodes a 147 amino acid protein including an 18 amino acid signal peptide for entrance into the secretory pathway of eukaryotic cells (FIG. 1).

The general methods and vectors for expressing heterologous proteins in plant cells in accordance with the present invention are described in the following patents of the intended assignee of the present application, which are hereby incorporated by reference in their entirety: U.S. Pat. Nos. 5,316,931; 5,589,367; 5,811,653; 5,922,602; 5,997,438; 5,965,794; 5,889,191; 6,054,566; 5,889,190; 5,866,785; 5,491,076; and 5,766,885.

U.S. Pat. Nos. 5,422,108 and 5,850,025, noted above, are also incorporated by reference.

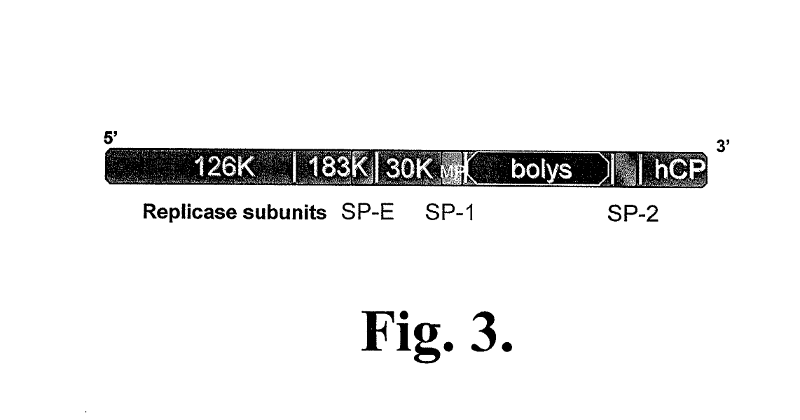

The p1044 BoLys vector is a virus expression system based on the tobacco mosaic virus (TMV) genome and contains a polylinker, a duplicated, non-native, subgenomic promoter and coat protein gene. A schematic map of the construct is shown in FIG. 3. The p1044 BoLys vector was then transcribed using T7 RNA polymerase and RNA cap analogue.

The resulting transcripts are preferably manually inoculated onto Nicotiana benthamiana plants. Seven days post inoculation, plants exhibited leaf crinkling and puckering indicative of a systemic infection.

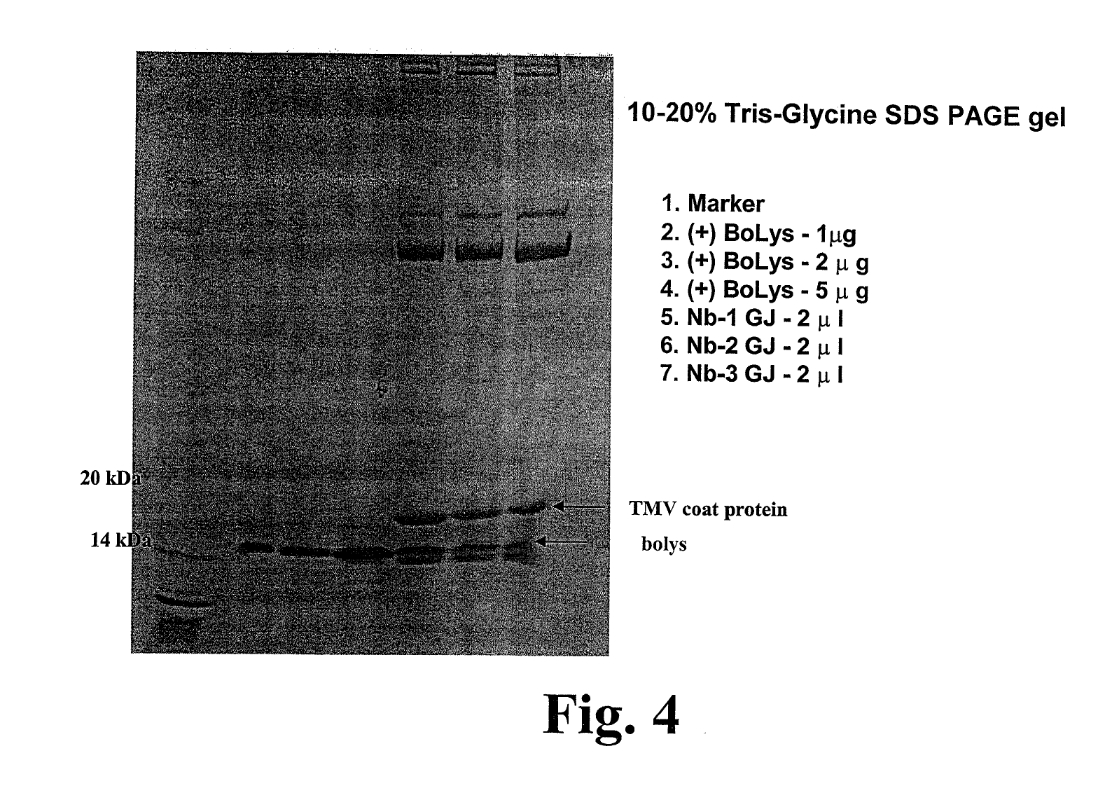

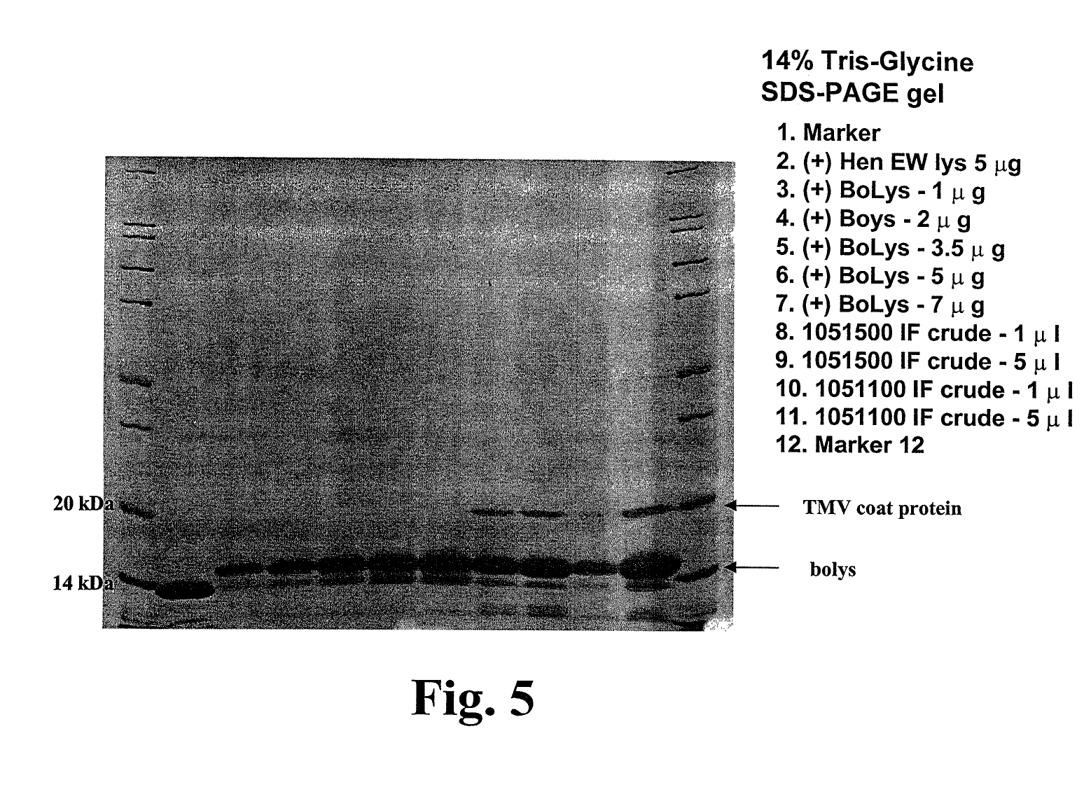

Plants 10 days post inoculation (dpi) were then harvested and subjected to two types of extractions (as described in the Examples). A portion of the plant materials was vacuum infiltrated and centrifuged to remove apoplastic (extra-cellular matrix) fraction of secreted proteins. A second portion of the plant was ground in an aqueous buffer and then centrifuged at ˜6,000× g to obtain a profile of the entire set of soluble plant proteins. The protein extracts were then separated on a SDS-PAGE gel to view proteins by molecular weight and stained with Coomassie blue. The gel showing expression pattern of bolys in total soluble proteins is shown in FIG. 4 and those from IF or infiltrated leaf extracts is shown in FIG. 5. Identity of the bolys protein band was confirmed using protein immunoblotting techniques with antibody preparations provided by NewGene.

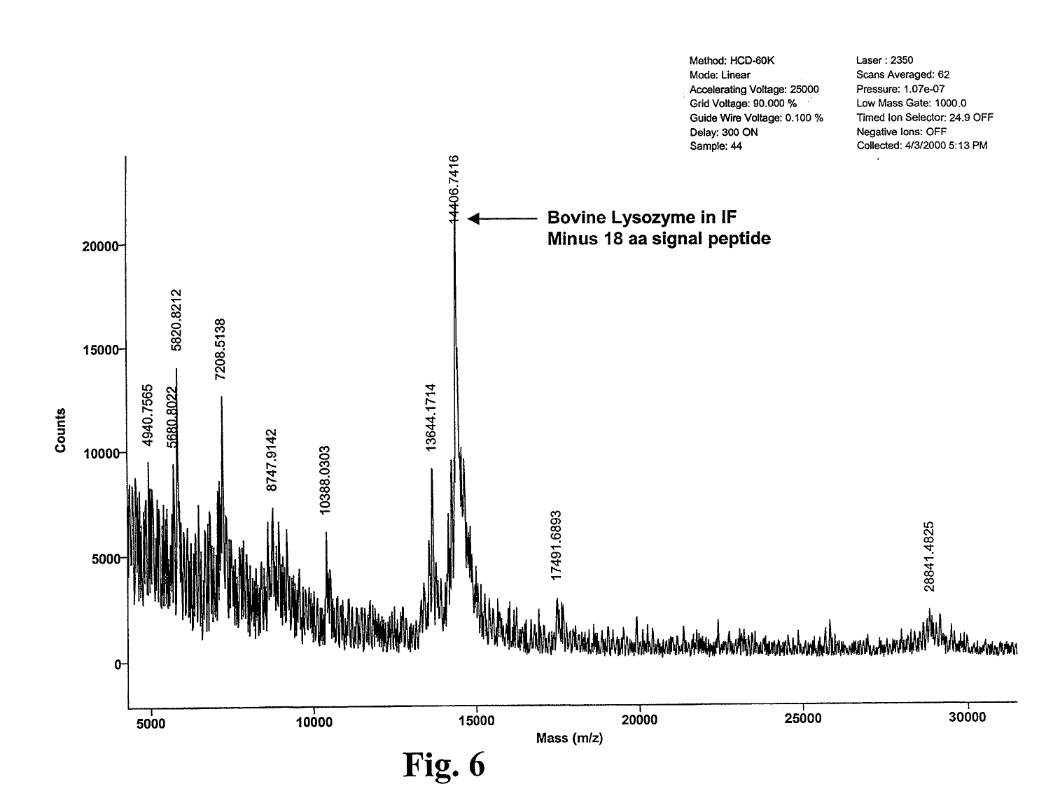

The protein extracts were tested for the molecular size of the bolys protein using mass spectroscopy (MALDI-TOF). The resulting size was 14,406 daltons, which matches with the size predicted from the primary amino acid sequence of the gene, lacking the signal peptide and protonated (FIG. 6). The protein was not glycosylated, or modified in any other manner indicating the plant system was processing the bovine protein exactly as would be predicted. Extracts were subjected to a variety of pH extremes, from pH 4.8 to 12.5 and the bolys protein proved stable. IF was also stored at 4° C. for days, weeks and months and analyzed for protein stability using SDS-PAGE. The protein appeared very resistant to plant proteases and did not diminish in quantity or quality (band integrity) over time.

Purification

Purification at scale offers several benefits and allows simplification of the methodology. Initial extractions of the protein from the plant tissue showed that >60% of the protein could be obtained through an IF procedure. However, a significant amount of material could be recovered through total plant homogenization. Whereas, IF extracts are largely devoid of protein content, save the TMV coat protein and any GENEWARE produced protein (FIG. 4), total homogenates contain large quantities of all soluble plant proteins. A range of pH and temperature steps was tested to find a set of conditions that rendered the bolys protein soluble, and eliminate the majority of the major contaminating proteins. Final conditions of pH 4.8 and heat treatment of 5 minutes at 45° C. were established to accomplish this purpose. These methods are detailed in LSBC patents and proved successful to loose less than 0.1% of the total bolys protein. The clarified extract now contains contaminating virus and the product as the major constituents. For USDA and CDFA movement purposes, the final preparation of bolys protein must be devoid of infectious, recombinant virus. Therefore, methods to separate the two components were developed.

The goal of this project was to develop a method that would work at full-scale for production. Therefore, chemical methods to separate the virus from the bolys protein, although very successful, were not used for the final process in lieu of physical separation methods. The soluble solution following pH and heat treatment was then passed through a 100 kDa ultrafiltration membrane. Western blotting of protein extracts and Coomassie staining of proteins showed the bolys protein passing through the membrane freely and provided a strong prediction of very high recovery when additional diafiltration steps are added to the process. The 100 kDa permeate (proteins passing through the 100 kDa membrane) that contained the bolys protein also showed no detectable virus or virus proteins.

In order to test the actual reduction in infectious units after various purification steps, infectivity assays were developed using Nicotiana tabacum cv. Xanthi NN hosts which show local necrotic lesion development upon replication of the TMV virus. The sensitivity of these assays was quantitated to be 20 ng of virus/ml inoculum. Infectivity tests were carried out on the 100 kDa permeate. These studies indicate that the 100 kDa-ultrafiltration step effectively removes all detectable infectious virus from the plant extracts. This solution can now be concentrated using a 3 kDa ultrafiltration unit.

These studies demonstrated that the bolys protein was effectively retained by the 3 kDa cutoff membrane and that no detectable bolys protein passed through the membrane. Again, no detectable coat protein was observed in the 3 kDa concentrate. These data indicate that the combination of lack of Coomassie staining of coat protein and lack of infectivity indicate efficient and functionally complete removal of all infectious particles from the extract.

This purity facilitates the movement of purified materials between states and within the state of California for testing of the enzyme.

At present, the following techniques, pH/heat treatment with centrifugation (clarification), 100 k ultrafiltration and diafiltration and 3 k ultrafiltration are preferred for the production of the target 100 g lot of bolys protein. The final purity, estimated at ˜80%, is achieved without chromatography steps. The yield of bolys protein using analogous purification procedures from several large lots of Nicotiana plants is between 0.5-1 mg per gram fresh weight of plant tissue.

Enzyme Activity

The activity of the bolys was tested to evaluate the ability of the protein to lyse actual bacteria. Two assays were developed to measure the enzymatic activity of the enzyme produced in plant systems: (1) a Micrococcus luteus turbidimetric assay and (2) a fluorescence assay for carbohydrate cleavage. Details are provided below.

1. Turbidimetric Assay

The lysozyme substrate is Micrococcus luteus. The lysozyme lyses the bacteria in the presence of sodium ions, which results in a decrease in absorbance at 450 nm, since lysed cells scatter less light than intact cells. The bacterial cells are suspended in a sodium phosphate buffer, pH 5. One ml of the cell suspension is added to the lysozyme-containing sample in a cuvette, which is immediately put in a spectrophotometer. The decrease in absorbance is measured at 30 second intervals for 2 minutes at 450 nm.

2. Fluorometric Assay

The lysozyme substrate used in this assay is 4-methylumbelliferyl-B-D-N, N′-N″ triacetylchitotriose. The lysozyme cleaves the substrate, releasing the methylumbelliferone, a substance that is excited at 360 nm and fluoresces at 460 nm. The lysozyme sample is incubated with the substrate in a pH 5 buffer for 1 hour at 42° C., and the reaction is stopped using a high pH buffer. Fluorescence is read in a fluorescent plate reader.

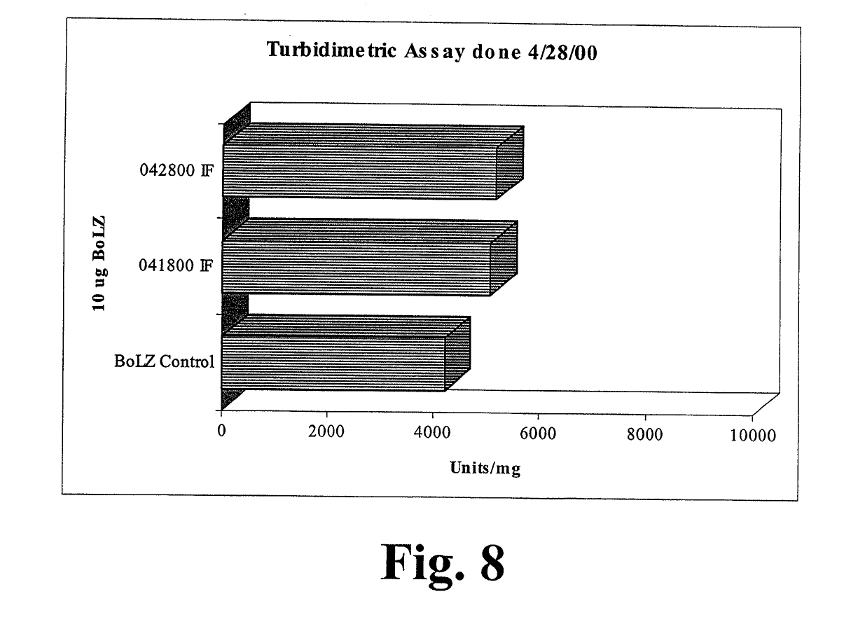

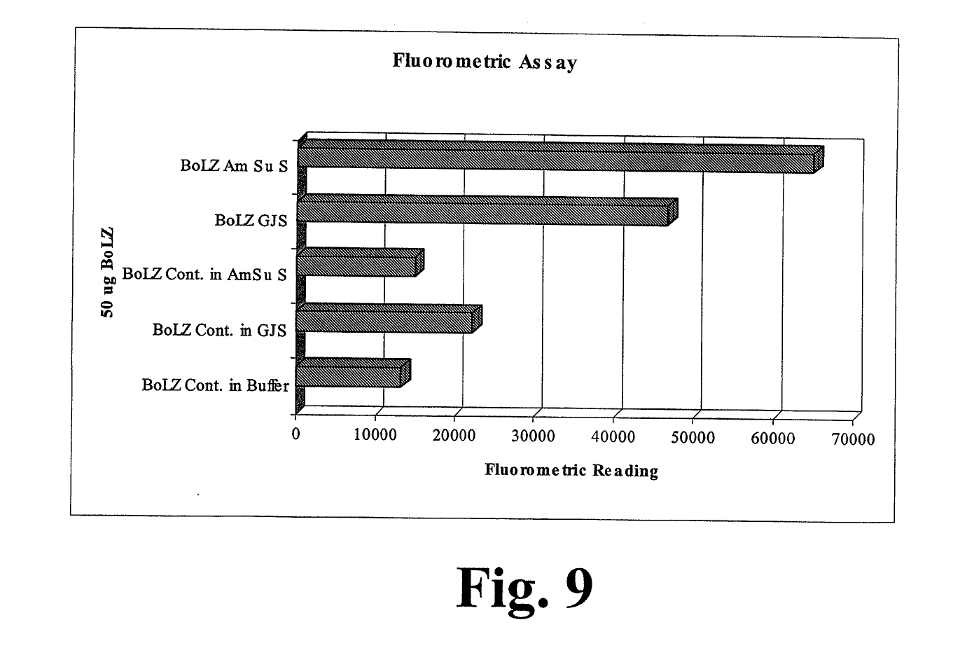

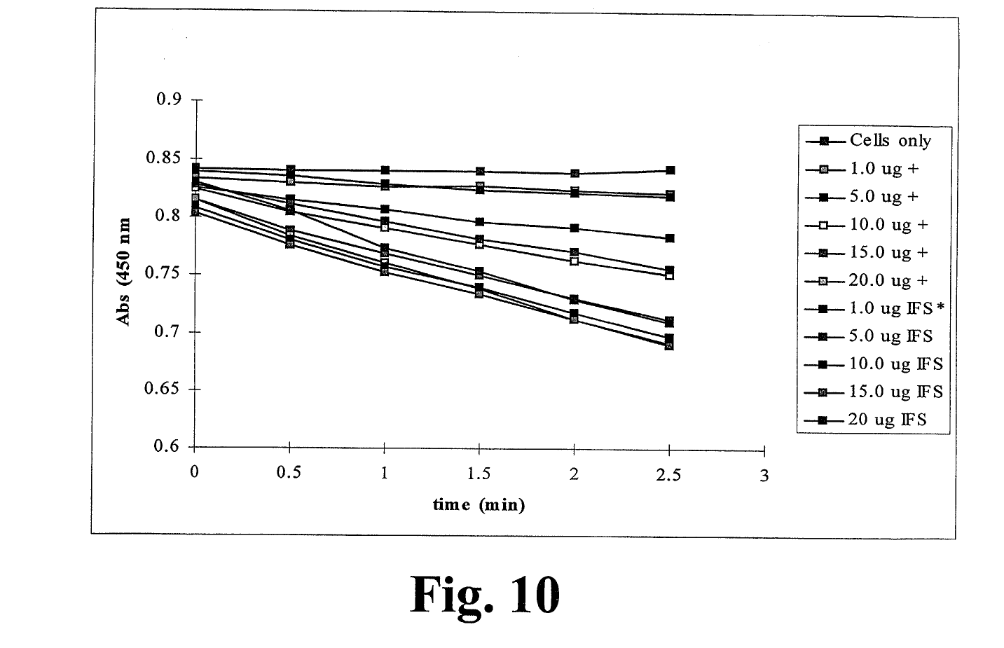

These experiments have employed a series of control enzymes, bovine lysozyme provided by NewGene, bovine and egg lysozyme from Sigma and various spiking procedures. Each assay measures distinct activities of the enzyme. The turbidimetric assay is quite specific for the function of interest, but it is quite insensitive to protein concentration. The fluorimetric assay is more sensitive, however can only provide a surrogate endpoint. The conclusion from many experiments using crude and purified bolys protein derived from GENEWARE® infected plants is that the enzyme activity of plant-produced bolys matches or exceeds that of Pichia-produced enzyme an that purchased from Sigma. (See FIG. 8.) These data suggest that this enzyme is capable of all predicted anti-microbial activities.

The question that remains to be resolved is whether the bolys protein will lyse and kill the grape variety of Xylella fastidiosa.

To initiate tests of killing of X. fastidiosa, the inventors obtained CDFA approval for samples of the grape variety of Xylella from Dr. Edward Civerolo at UC Davis, Dept of Plant Pathology. Xylella samples are moved to greenhouses for testing purposes. The Xylella is used for testing the activity of bolys protein by turbidimetric assay.

The inventors are establishing a Nicotiana-based system for Xylella infection. This allows many surrogate tests including virus delivery of effective doses of bolys protein and other protein-based delivery approaches. It has been discovered that PD-inducing strain of Xylella established a xylem infection in Nicotiana (Kava et al., Phytopath. 89:S33).

Production

Full-scale production of bolys protein for testing purposes is at the Owensboro site of LSBC. The bolys protein is also manufactured in greenhouse grown plants in addition to, or instead of, field plants. The greenhouse space houses plants for production runs to obtain ˜100 g of purified bolys protein. Runs from 12,000 Nicotiana plants are be used to optimize purification procedures and produce the product.

A three stage process is preferably employed a bench scale 1-5 kg process is performed to optimize purification steps. On the following week, pilot scale optimization will be completed on 150 kg batches; the third week, Sep. 18, 2000, 4 days of full-scale “pilot” manufacturing of bolys protein from 50 kg/day are carried out to complete the process. Formulation and testing continues in the following week.

To test the efficacy of bolys protein against xylem-limited bacteria begin in short order. Xylella grows very slowly and generally loses pathogenicity if it is passaged more than twice. The initial goal is to establish liquid cultures of Xylella. Turbidimetric assays are designed to test the ability and efficacy of plant derived and control bolys protein to kill Xylella in vitro.

The Lopez et al., 2000 (Plant Disease 84, 827) and Phytopathology (supra) abstracts that indicate the citrus strain and PD strain of Xylella can infect Nicotiana tabacum cultivars will prompt a thorough development of a more rapid test system for the grape pest. Various Nicotiana species and cultivars including RP1 will be tested for susceptibility to Xylella and if successful, a model system may be ready for our collaboration.

Expression of Bolys Protein

The expression of the bovine lysozyme protein may be driven by any of a variety of promoters functional in the context of the recombinant plant viral vector and host plant. In a preferred embodiment plant viral subgenomic promoters are used (U.S. Pat. No. 5,316,931).

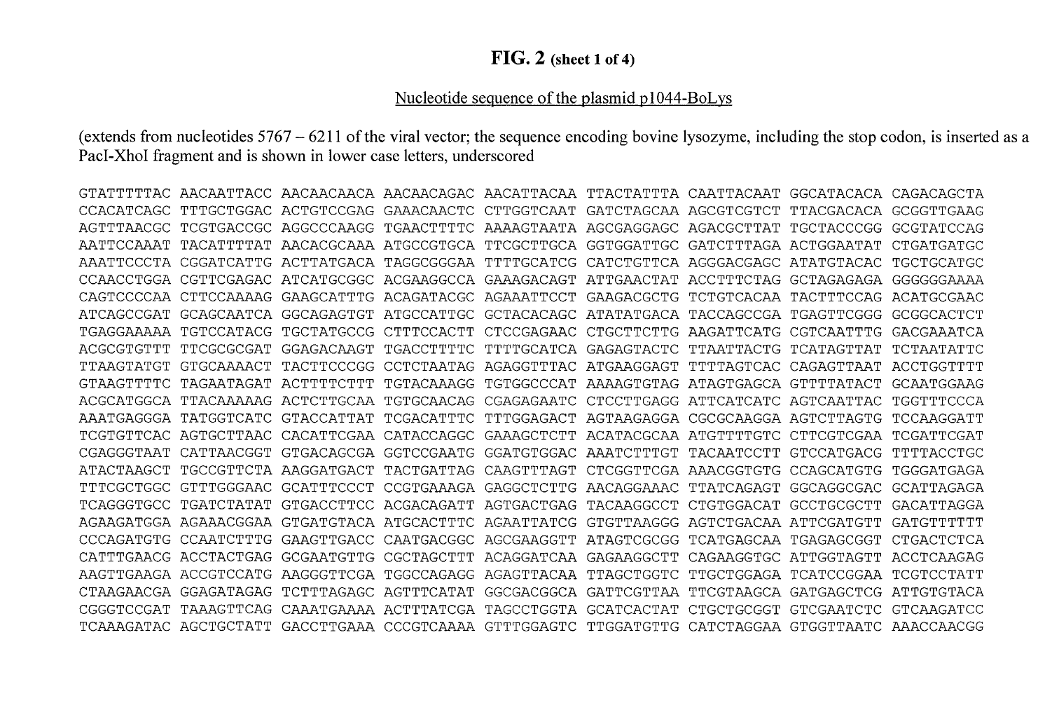

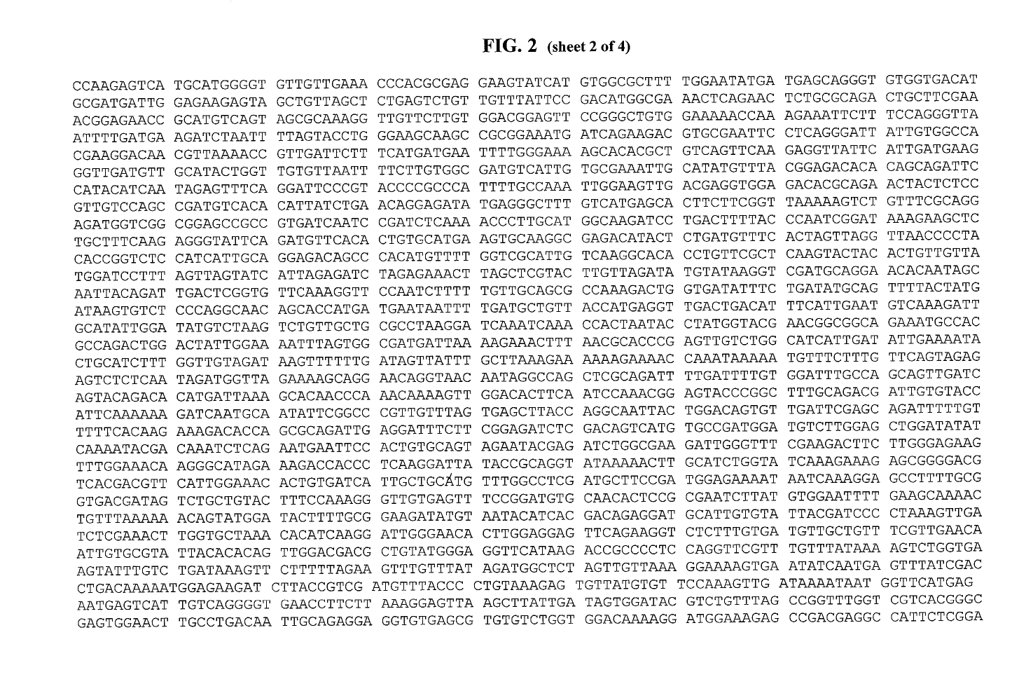

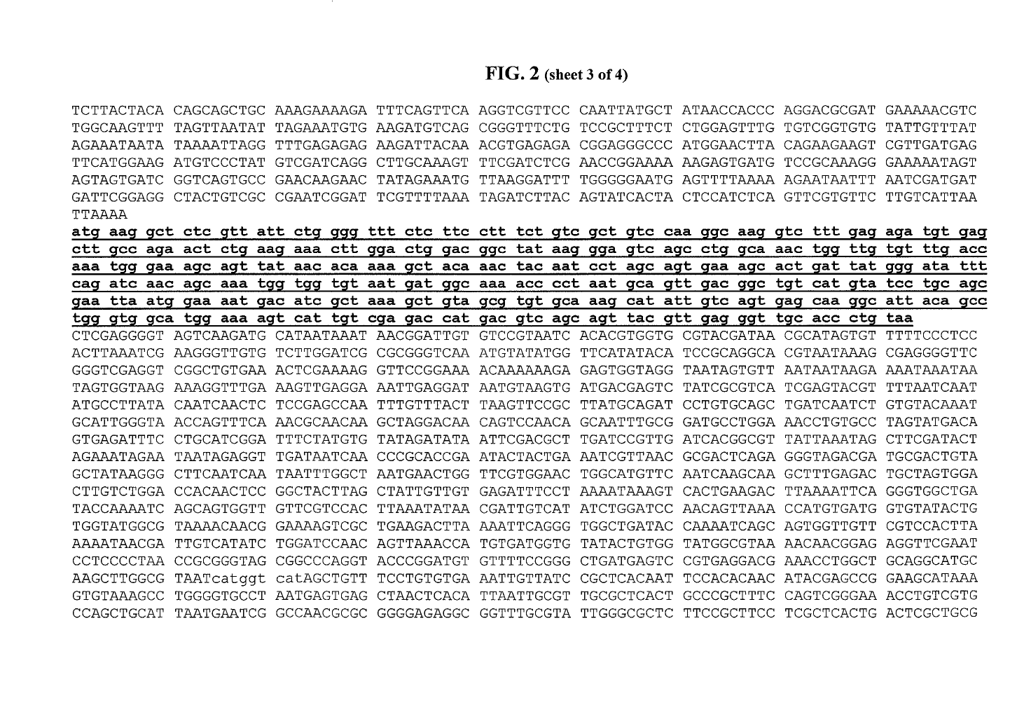

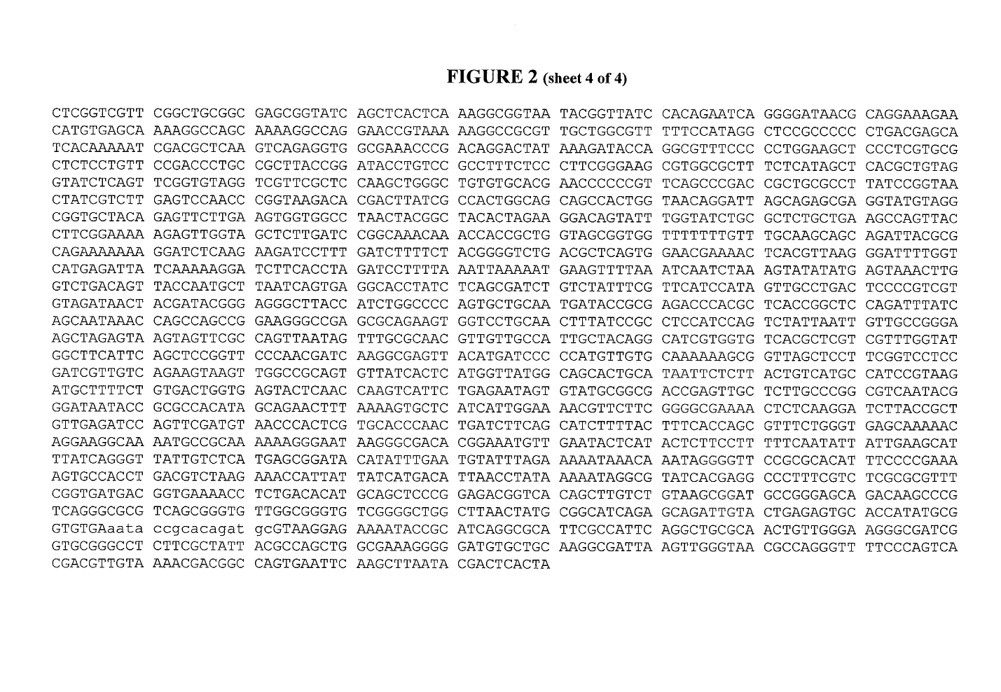

The p1044 BoLys vector is a virus expression system based on the tobacco mosaic virus (TMV) genome and contains a polylinker, a duplicated, non-native, subgenomic promoter and coat protein gene. The general map of the construct is shown in FIG. 2. The p1044 BoLys vector was then transcribed using T7 RNA polymerase and RNA cap analogue and the resulting transcripts were then manually inoculated onto Nicotiana benthamiana plants. Seven days post inoculation, plants exhibited leaf crinkling and puckering indicative of a systemic infection. Plants 10 days post inoculation (dpi) were then harvested and subjected to two types of extractions. A portion of the plant materials was vacuum infiltrated and centrifuged to remove apoplastic (extra-cellular matrix) fraction of secreted proteins. A second portion of the plant was ground in an aqueous buffer and then centrifuged at 6,000× g to obtain a profile of the entire set of soluble plant proteins. The protein extracts were then separated on a SDS-PAGE gel to view proteins by molecular weight and stained with Coomassie blue. The gel showing expression pattern of bolys in total soluble proteins is shown in FIG. 3 and those from IF or infiltrated leaf extracts is shown in FIG. 4. Identity of the bolys protein band was confirmed using protein immunoblotting techniques with antibody preparations provided by NewGene.

The protein extracts were tested for the molecular size of the bolys protein using MALDI-TOF spectroscopy. The resulting size was 14,406 daltons, which matches with the size predicted from the primary amino acid sequence of the gene, lacking the signal peptide and protonated (FIG. 5). The protein was not glycosylated, or modified in any other manner indicating the plant system was processing the bovine protein exactly as would be predicted. Extracts were subjected to a variety of pH extremes, from pH 4.8 to 12.5 and the bolys protein proved stable. IF was also stored at 4° C. for days, weeks and months and analyzed for protein stability using SDS-PAGE. The protein appeared very resistant to plant proteases and did not diminish in quantity or quality (band integrity) over time.

Recombinant DNA technologies have allowed the life cycle of numerous plant RNA viruses to be extended artificially through a DNA phase that facilitates manipulation of the viral genome. These techniques may be applied by the person of ordinary skill in the art to make and use recombinant plant viruses of the invention. The entire cDNA of the TMV genome was cloned and functionally joined to a bacterial promoter in an E. coli plasmid (Dawson, W. O. et al., Proc. Natl. Acad. Sci. USA 83:1832-1836 (1986)). Infectious recombinant plant viral RNA transcripts may also be produced using other well known techniques, for example, commercially available RNA polymerases from T7, T3 or SP6. Precise replicas of the virion RNA can be produced in vitro with RNA polymerase and dinucleotide cap, m7GpppG. This not only allows manipulation of the viral genome for reverse genetics, but it also allows manipulation of the virus into a vector to express foreign genes. A method of producing plant RNA virus vectors based on manipulating RNA fragments with RNA ligase has proved to be impractical and is not widely used (Pelcher, L. E. et al., EP 67553A2 (1982). Detailed information on how to make and use recombinant RNA plant viruses can be found, among other places, in U.S. Pat. No. 5,316,931, which is herein incorporated by reference. The invention provides nucleic acids that comprise a recombinant RNA plant vector for expression of the subject fusion proteins. The invention also provides for nucleic acids that comprise a portion or portions of the subject vectors. The vectors described in U.S. Pat. No. 5,316,931 are particularly preferred for expressing the fusion proteins of the invention.

The invention also provides recombinant plant cells comprising the subject lysozyme proteins and/or virus particles comprising nucleic acid encoding the subject proteins. These plant cells may be produced either by infecting plant cells (in culture or in whole plants) with the infectious recombinant virus particles of the invention or with polynucleotides comprising the genomes of the infectious virus particle of the invention. The recombinant plant cells of the invention have many uses, among which is serving as a source for the lysozyme proteins of the invention.

Having now generally described the invention, the same will be more readily understood through reference to the following examples which are provided by way of illustration, and are not intended to be limiting of the present invention, unless specified.

### EXAMPLE 1

### Vector Construction and Plant Growth

A nucleic acid molecule encoding bovine lysozyme, shown in FIG. 1, was cloned behind a subgenomic promoter in a Tobamovirus having dual heterologous subgenomic promoters. The resulting virus, p1044-BoLys, ATCC Dep. No. PTA-2599, deposited on Oct. 16, 2000 (and having the sequence SEQ ID NO:3), was rubbed onto the leaf of a young Nicotiana benthamiana plant. The plant was grown under controlled greenhouse conditions.

### EXAMPLE 2

### Lysozyme Isolation

Extraction and Purification of Bovine Lysozyme, pH-Heat Extraction

Nicotiana benthamiana produced in greenhouses, were inoculated with a tobacco mosaic virus vector expressing bovine lysozyme. Plants were harvested 2-3 weeks post inoculation after systemic spread of the virus. Leaf and stalk tissue (50-65 kg) was disrupted by two passes through a Corenco disintegrator (Model M8A-D) with the addition of 50-65 L of chilled, 0.04% Na₂S₂O₅. The initial cell disruption was performed using knives and a 1/2 inch, square hole screen. The second disintegration was performed by passing the initial homogenate through a hammer stack and 3/16 inch, round hole screen. The macerated material was pressed through a Vincent Horizontal Screw press containing a 0.023 inch perforated screen (Model VP6K28) to remove fibrous material. The resultant “green juice” was adjusted to a pH of 5.0 with H₃PO4. The pH adjusted green juice was heated to 47° C. by passage through an Alfa-Laval heat exchanger (Model M3VG) and held at this temperature for 5-10 minutes. The green juice was then cooled to 10-15° C. by passage through an Alfa-Laval heat exchanger (Model M3VG). Solid ammonium sulfate was added to the heat-treated green juice at a concentration of 15-20% saturation, dissolved by stirring and incubated for 30 minutes. Ammonium sulfate treated green juice was centrifuged through a Westfalia Disk Stack centrifuge (Model SA7-06-076) resulting in two fractions, supernatant 1 and pellet 1. The supernatant 1 fraction was centrifuged by two passes through a Sharples, AS-26 centrifuge (17,000 RPM) at a feed rate of 1.0 L per minute. Bovine lysozyme was separated from residual TMV by ultrafiltration through an Amicon, 40 square foot, cellulose acetate, spiral membrane having a 100 kDa molecular weight cut-off. Bovine lysozyme passed through the 100 kDa membrane and was recovered from the permeate fraction while TMV was retained by the membrane. The bovine lysozyme present in the 100 kDa permeate was concentrated using an Amicon, 40 ft², cellulose acetate, spiral membrane having a 3 kDa molecular weight cut-off. After concentration, ammonium sulfate was removed from the lysozyme present in the 3 kDa retentate by diafiltration with water or buffer.

### EXAMPLE 3

### Turbidimetric Assay of Bovine Lysozyme Enzymatic Activity

This assay was conducted to determine the activity of bovine lysozyme produced at the Owensboro facility and compare it to the activity of Pichia bovine lysozyme standard.

Samples:

| Sample # | Sample ID | dil. | # μl dil. | # μl undil. |
|---|---|---|---|---|
| 1 | BoLZ standard | — | — | 10 |
| 2 | 3K-1:1 | 1/10 | 20 | 2 |

Procedure:

1. Performed BCA assay to get an idea of relative concentrations of protein in samples.

2. Ran samples on SDS-PAGE gels to determine actual concentration of bovine lysozyme

3. Diluted samples, aliquoting appropriate amounts of each sample into eppendorf tubes. Added diluent to a volume of 20 μl. Prepared duplicates of each sample.

4. Centrifuged M. luteus cultures (grown at room temperature). Rinsed pellets once in 100 mM NaH₂PO₄, pH 5.0.

5. Resuspended pellet in buffer to final OD of 0.75.

6. Added 1 ml of cell suspension to sample, transferred to a cuvette, and inserted into spectrophotometer.

7. Read decrease in OD at 450 nm for 2 minutes at 30 second intervals.

Results and Conclusion

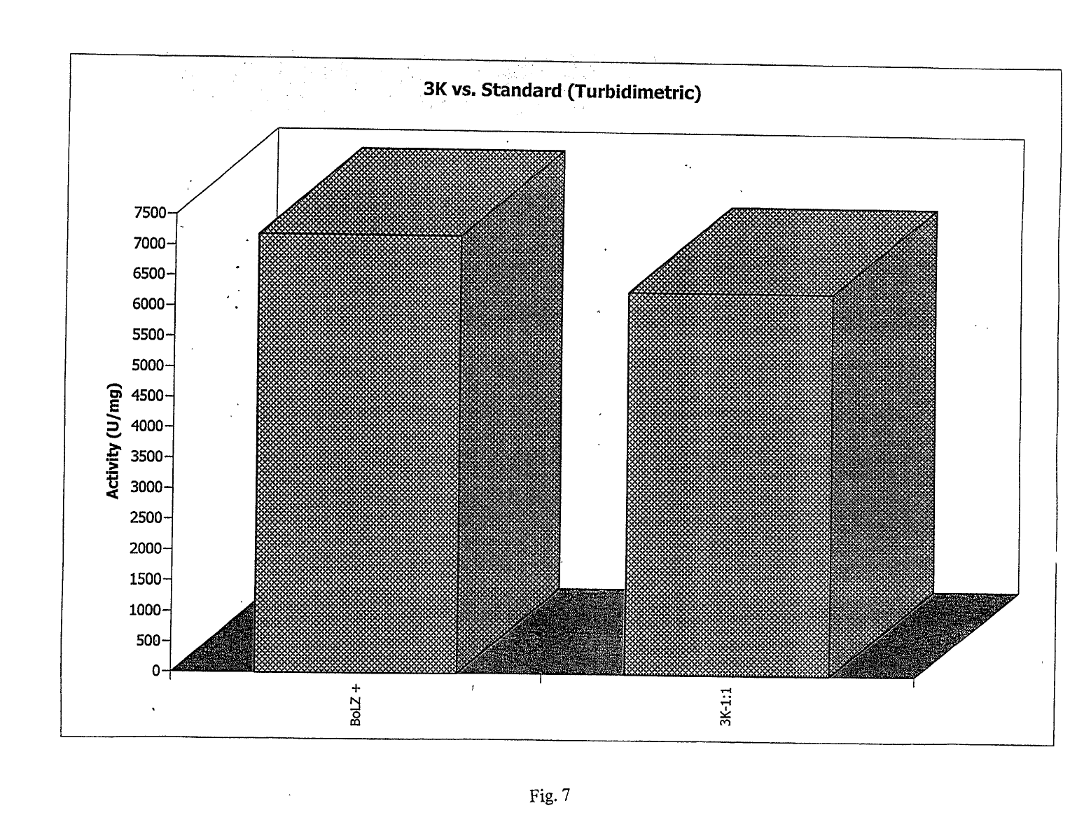

The results are shown in FIG. 7. The Owensboro-produced bovine lysozyme had very similar activity to the Pichia bovine lysozyme, about 87.5% of the activity of the lysozyme standard.

The references cited above are all incorporated by reference herein, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth as follows in the scope of the appended claims.

What is claimed is:

1. A plasmid capable of expressing a heterologous protein in a plant that encodes bovine lysozyme.

2. The plasmid of claim 1 comprising SEQ ID NO: 1.

3. The plasmid of claim 1 or 2 having the sequence SEQ ID NO:3.

4. The plasmid of claim 3 identified as ATCC Dep. No. PTA-2599.

5. A recombinant RNA plant virus comprising a nucleotide sequence encoding bovine lysozyme.

6. The plant virus of claim 5 comprising SEQ ID NO: 1.

7. An RNA molecule comprising:
(a) a first viral subgenomic promoter;
(b) a second viral subgenomic promoter; and
(c) a bovine lysozyme coding sequence under control of either the first or the second subgenomic promoter.

8. The RNA molecule of claim 7 wherein the coding sequence is SEQ ID NO:1.

9. A recombinant tobamovirus comprising a nucleotide sequence encoding bovine lysozyme.

10. The tobamovirus of claim 9 wherein the lysozyme coding sequence is SEQ ID NO:1.

11. A method of producing bovine lysozyme, comprising:
(a) infecting a host plant with an RNA plant virus comprising a nucleotide sequence encoding bovine lysozyme;
(b) allowing the virus to infect the plant systemically;
(c) harvesting plant material; and
(d) isolating from said harvested plant material bovine lysozyme, thereby producing said bovine lysozyme.
